(19) United States
(12) Patent Application Publication
(10) Pub. No.: US 2008/0095304 A1
(43) Pub. Date: Apr. 24, 2008

(54) Energy-Resolved Computer Tomography

(75) Inventors: Michael Grass, Buchholz In Der Nordheide (DE); Thomas Koehler, Norderstedt (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/718,719

(22) PCT Filed: Nov. 2, 2005
(86) PCT No.: PCT/IB05/53564

§ 371 Date: May 7, 2007

(30) Foreign Application Priority Data
Nov. 11, 2004 (GB) 0424876.1

(51) Int. Cl. A61B 6/03

(52) U.S. Cl. 378/6

(57) ABSTRACT

In coherent scatter computed tomography, the scatter angle from object points (31, 32, 33, 34, 35), which are located on a line (36) perpendicular to the central beam (45) in the plane of rotation, varies as a non-linear function of the fan-angle of the detector row (37). According to an exemplary embodiment of the present invention, a single-line energy-resolved detector for CSCT data acquisition is used, which measures scattered photons originating from object points on a circular arc under the same scatter angle. This automatically leads to a Cartesian q-sampling on a parallel-rebinned detector. Advantageously, this may avoid q-interpolation prior to parallel-rebinned filtered back-projection reconstruction.

The present invention relates to the field of computer tomography. In particular, the present invention relates to a computer tomography apparatus for examination of an object of interest, to a radiation detector, to a method of examination of an object of interest in a computer tomography apparatus and to a computer program for performing an examination of an object of interest in to a computer tomography apparatus.

Over the past several years, x-ray baggage inspections or medical applications have evolved from simple x-ray imaging systems that were completely dependent on an interaction by an operator to more sophisticated automatic systems that can automatically recognize certain types of materials. An inspection system has employed an x-ray radiation source for emitting x-rays which are transmitted through or scattered from the examined package to a detector.

An imaging technique based on coherently scattered x-ray photons is the so-called “Coherent Scatter Computer Tomography” (CSCT). CSCT is a technique that produces images of the low angle scatter properties in an object of interest. These depend on the molecular structure of the object, making it possible to produce material-specific maps of each component. The dominant component of low angle scatter is coherent scatter. Since coherent scatter spectra depend on the atomic arrangement of the scattering sample, coherent scatter computer tomography is a sensitive technique for imaging spatial variations in the molecular structure of baggage or biological tissue across a two-dimensional object section.

A CSCT system is built of an x-ray tube, illuminating one slice of the object, and a detection system, both rotating around the object of interest. The detector system may either be a two-dimensional detector, which measures the off-plane scattered photons, or a single-row detector, which performs an energy-resolved measurement of the scattered photons. From the measured protection data, a three-dimensional volume is reconstructed defined by the two spatial dimensions (x, y) in the plane of primary radiation. The third dimension is parameterized by the momentum transfer q of the scattered photons. In case that an energy-resolved focus-centred single-row detection system with a fixed distance H from the plane of rotation is used, the scatter angle from object points, which are located in a line perpendicular to the central beam in the plane of rotation, varies as a non-linear function of the fan-angle β of the detector row.

Thus, typically a q-interpolation prior to a filtered back-projection reconstruction has to be performed, which requires additional computational effort and may reduce the resolution in q and therefore the image quality.

Hence, there is a desire for the provision of an improved examination of an object.

According to an exemplary embodiment of the present invention, a computer tomography apparatus for examination of an object of interest is provided, the computer tomography apparatus comprising a rotating source of electromagnetic radiation emitting a beam of electromagnetic radiation to an object of interest, a first detecting element adapted for detecting electromagnetic radiation coherently scattered from a first object point of the object of interest under a first scatter angle and a second detecting element adapted for detecting electromagnetic radiation coherently scattered from a second object point of the object of interest under a second scatter angle, wherein the first object point and the second object point are positioned on a circular arc and wherein the first scatter angle equals the second scatter angle. The first detecting element and the second detecting element are part of a single-row energy-resolved detector.

Advantageously, according to this exemplary embodiment of the present invention, the scatter radiation detected by the first detecting element is scattered from the first object point under the same scatter angle as the scatter radiation which is scattered from the second object point and detected by the second detecting element. Therefore, data under the same scatter angle is acquired by the first and second detecting elements, which may improve the resolution in momentum-transfer and the computational efficiency in CSCT reconstruction.

According to another exemplary embodiment of the present invention, a first distance between the first detecting element and a plane of rotation of the rotating source is a predetermined function of a first fan-angle between the central ray and a ray emitted from the source to the first object point and to wherein a second distance between the second detecting element and the plane of rotation is a predetermined function of a second fan-angle between the central ray and a ray emitted from the source to the second object point.

Advantageously, since the distances between the detector elements and the plane of rotation are a function of the fan-angle (and of the position of each detector element in the plane of rotation (x, y-plane)), a single-row detector may be configured according to the specific function before the measurement starts.

According to another exemplary embodiment of the present invention, the computer tomography apparatus further comprises a data processor, wherein the data processor is adapted for performing the operation of linear sampling in an energy for each detecting element and applying a parallel-beam rebinning of the beam into a parallel beam geometry, which results in an equidistant sampling in an momentum transfer of the detected radiation for each detecting element without interpolation.

Thus, according to this exemplary embodiment of the present invention, using a detector with bending defined by a predetermined function, an equidistant sampling in q-direction automatically results when a linear sampling in the energy is used and a fan-beam to parallel-beam rebinning is applied.

According to another exemplary embodiment of the present invention, the first detecting element and the second detecting element are part of a radiation detector and the radiation detector is one of a focus-centred single-row energy-resolved detector and a planar single-row energy-resolved detector.

Advantageously, the use of a focus-centred or planar single line detectors may reduce the loss in resolution in q-direction since no interpolation in q-direction is performed.

According to another exemplary embodiment of the present invention, the source of electromagnetic radiation is a polychromatic x-ray source moving along a helical path around the object of interest, wherein the beam has a fan-beam geometry.

The application of a polychromatic x-ray source is advantageous, since polychromatic x-rays are easy to generate and provide a good image resolution.

The computer tomography apparatus may be adapted as a coherent scatter computer tomography apparatus (CSCT), i.e. a computer tomography apparatus may be configured and operated according to the CSCT technology described above.

A collimator may be arranged between the x-ray source and the first and the second detecting elements, the collimator being adapted to collimate an x-ray beam emitted by the x-ray source to form a fan-beam. A fan-beam is the preferred beam-shape of the CSCT technology. By implementing such a collimator preferably having an elongated slit, it may be possible to use almost any desired x-ray source, since a properly shaped collimator produces a fan-beam from any type of primary x-ray beam geometry.

The first detecting elements and the second detecting elements may be provided with a common casing. This may allow for a very compact configuration of the apparatus.

The x-ray tomography apparatus according to the invention may be configured as one of the group consisting of a baggage inspection apparatus, a medical application apparatus, a material testing apparatus and a material science analysis apparatus. However, the most preferred field of application of the invention is baggage inspection or medical applications, since the functionality of the invention allows a secure and reliable analysis of the object of interest.

According to another exemplary embodiment of the present invention, a radiation detector is provided comprising a first detecting element adapted for detecting electromagnetic radiation emitted from a source of electromagnetic radiation and coherently scattered from a first object point of an object of interest under a first scatter angle, and a second detecting element adapted for detecting electromagnetic radiation emitted from the source and coherently scattered from a second object point of the object of interest under a second scatter angle. The first object point and the second object point are positioned on a circular arc and the first scatter angle equals the second scatter angle, wherein the first detecting element and the second detecting element are part of a single-row energy-resolved detector.

According to this exemplary embodiment of the present invention, a radiation detector is provided which may allow for an energy-resolved coherent scatter computer tomography for baggage inspection or medical applications with improved spatial resolution, a reduction of computational effort and an improved image quality.

In the following, preferred embodiments of the methods of examining an object of interest with a computer tomography apparatus will be described. However, these embodiments also are applied for the computer tomography apparatus of the invention.

The method of the invention may further comprise the steps of rotating a source of electromagnetic radiation, emitting a beam of electromagnetic radiation from the source to an object of interest, detecting electromagnetic radiation coherently scattered from a first object point of the object of interest under a first scatter angle by a first detecting element and detecting electromagnetic radiation coherently scattered from a second object point of the object of interest under a second scatter angle by a second detecting element, wherein the first object point and the second object point are positioned on a circular arc and wherein the first scatter angle equals the second scatter angle, wherein the first detecting element and the second detecting element are part of a single-row energy-resolved detector.

The present invention also relates to a computer program, which may, for example, be executed on a processor, such as an image processor. Such a computer program may be part of, for example, a CSCT scanner system. The computer program may be preferably loaded into working memories of a data processor. The data processor is thus equipped to carry out exemplary embodiments of the methods of the present invention. The computer program may be written in any suitable programming language, such as, for example, C++ and may be stored on a computer-readable medium, such as a CD-ROM. Also, these computer programs may be available from a network, such as the WorldWideWeb, from which they may be downloaded into image processing units or processors, or any suitable computers.

One aspect of the present invention may be that a single-line energy-resolved detector for CSCT data acquisition is used, which measures scattered photon originating from object points on a line, which is perpendicular to the central ray of the original fan of x-rays, under the same scatter angle. This automatically leads to a Cartesian q-sampling on a parallel-rebinned detector. Advantageously, this may avoid q-interpolation prior to parallel-rebinned filtered back-projection reconstruction.

The aspects defined above and further aspects of the invention are apparent from the examples of embodiments to be described hereinafter and are explained with reference to these examples of embodiments.

Exemplary embodiments of the present invention will be described in the following, with reference to the following drawings:

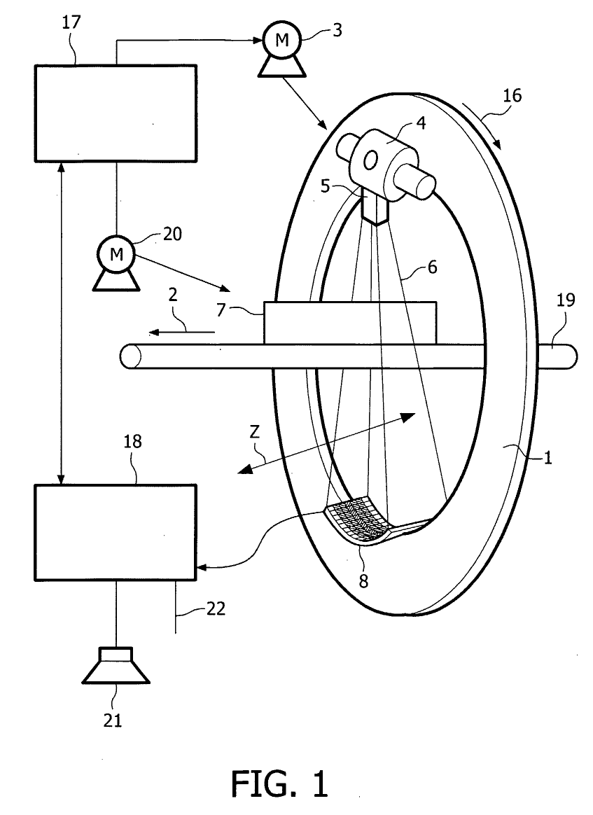

FIG. 1 shows a simplified schematic representation of an embodiment of a computer tomography scanner according to the present invention.

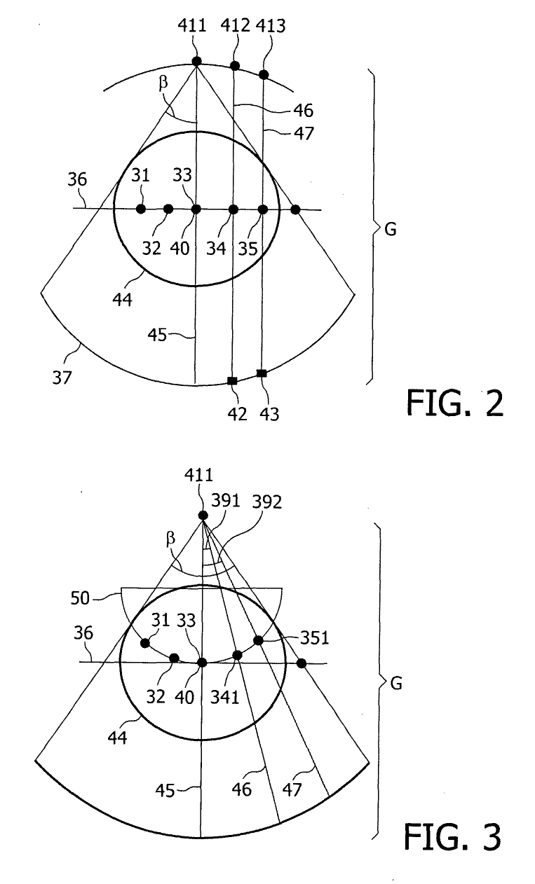

FIG. 2 depicts a schematic representation of a CSCT acquisition geometry along the rotational axis according to an exemplary embodiment of the present invention.

FIG. 3 depicts a schematic representation of the CSCT acquisition geometry of FIG. 2 in a fan-beam geometry.

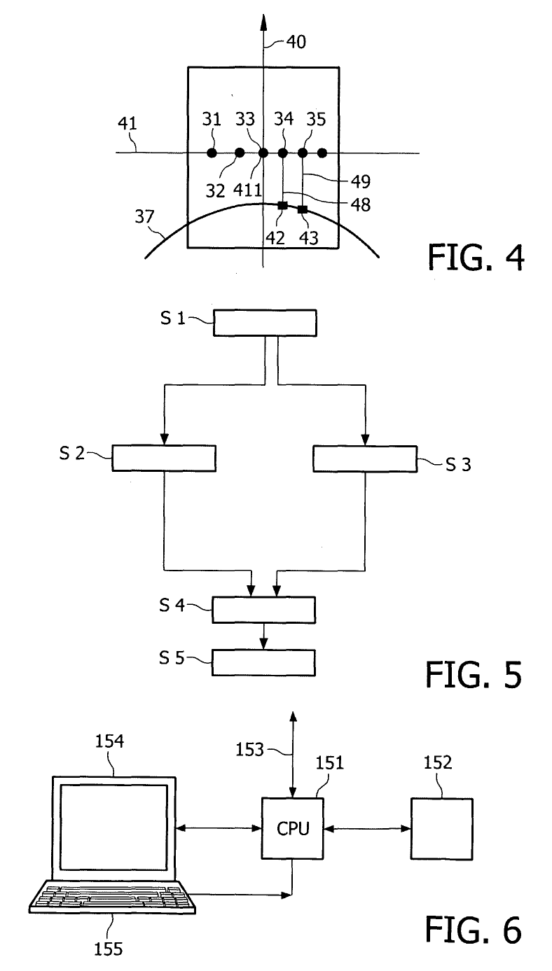

FIG. 4 shows a schematic representation of the CSCT acquisition geometry of FIG. 2 along an axis perpendicular to the rotational axis.

FIG. 5 shows a flow-chart of an exemplary embodiment of a method according to the present invention.

FIG. 6 shows an exemplary embodiment of an image processing device according to the present invention for executing an exemplary embodiment of a method in accordance with the present invention.

In different drawings, similar or identical elements are provided with the same reference numerals.

In the following, referring to FIG. 1, a computer tomography apparatus will be described having implemented energy-resolved CSCT.

With reference to this exemplary embodiment, the present invention will be described for the application in medical imaging. However, it should be noted that the present invention is not limited to the application in the field of medical imaging, but may be used in applications such as baggage inspection to detect hazardous materials, such as explosives, in items of baggage or other industrial applications, such as material testing.

The scanner depicted in FIG. 1 is a fan-beam CT scanner. The CT scanner depicted in FIG. 1 comprises a gantry 1, which is rotatable around a rotational axis 2. The gantry 1 is driven by means of a motor 3. Reference numeral 4 designates a source of radiation, such as an x-ray source, which, according to an aspect of the present invention, emits a polychromatic radiation beam.

Reference numeral 5 designates an aperture system which forms a radiation beam emitted from the radiation source to a cone-shaped radiation beam 6. After emitting a cone-shaped radiation beam 6, the beam may be guided through a slit collimator (not shown in FIG. 1) to form a primary fan-beam impinging on an object 7 to be located in an object region.

The fan-beam 6 (which in FIG. 1 is represented in an exaggerated manner; in reality it may only impinge on the central row of detecting elements, if not scattered along it's path) is now directed such that it penetrates the object of interest 7 arranged in the center of the gantry 1, i.e. in an examination region of the CSCT scanner and impinges onto the detector 8. As may be taken from FIG. 1, the detector 8 is arranged on the gantry 1 opposite the source of radiation 4, such that the surface of the detector 8 is covered by the fan-beam 6. The detector 8 depicted in FIG. 1 comprises a plurality of detector elements.

During a scan of the object of interest 7, the source of radiation 4, the aperture system 5 and detector 8 are rotated along the gantry 1 in the direction indicated by arrow 16. For rotation of the gantry 1 with the source of radiation 4, the aperture system 5 and the detector 8, the motor 3 is connected to a motor control unit 17, which is connected to a calculation unit 18.

During a scan, the radiation detector 8 is sampled at predetermined time intervals. Sampling results read from the radiation detector 8 are electrical signals, i.e. electrical data, which are referred to as projection in the following. A whole data set of a whole scan of an object of interest therefore consists of a plurality of projections where the number of projections corresponds to the time interval with which the radiation detector 8 is sampled. A plurality of projections together may also be referred to as volumetric data. Furthermore, the volumetric data may also comprise electrocardiogram data.

In FIG. 1, the object of interest is disposed on a conveyor belt 19. During the scan of the object of interest 7, while the gantry 1 rotates around the patient 7, the conveyor belt 19 displays the object of interest 7 along a direction parallel to the rotational axis 2 of the gantry 1. By this, the object of interest 7 is scanned along a helical scan path. The conveyor belt 19 may also be stopped during the scans. Instead of providing a conveyor belt 19, for example, in medical applications, where the object of interest 7 is a patient, a movable table may be used. However, it should be noted that in all of the described cases it is also possible to perform a circular scan, where there is no displacement in a direction parallel to the rotational axis 2, but only the rotation of the gantry 1 around the rotational axis 2.

The detector 8 is connected to the calculation unit 18. The calculation unit 18 receives the detection result, i.e. the read-outs from the detector element of the detector 8, and determines a scanning result on the basis of the read-outs. The detector elements of the detector 8 may be adapted to measure the attenuation caused to the fan-beam 6 by the object of interest 7 or the energy and intensity of x-rays coherently scattered from an object point of the object of interest 7 with an energy inside a certain energy interval. Furthermore, the calculation unit 18 communicates with the motor control unit 17 in order to coordinate the movement of the gantry 1 with motor 3 and 20 or with the conveyor belt 19.

The calculation unit 18 may be adapted for reconstructing an image from read-outs of the detector 8. The image generated by the calculation unit 18 may be output to a display (not shown in FIG. 1) via an interface 22.

The calculation unit 18 which may be realized by a data processor may also be adapted to perform an examination of an object of interest including the step of loading a data set acquired by means of a rotating source of electromagnetic radiation rotating in a plane of rotation and emitting a beam of electromagnetic radiation to an object of interest. The data set may comprise data detected by a first detecting element and data detected by a second detecting element, wherein the data detected by the first detecting element corresponds to electromagnetic radiation coherently scattered from a first object point of the object of interest under a first scatter angle and wherein the data detected by the second detecting element corresponds to electromagnetic radiation coherently scattered from a second object point of the object of interest under the same scatter angle.

Furthermore, as may be taken from FIG. 1, the calculation unit 18 may be connected to a loudspeaker 21 to, for example, automatically output an alarm.

FIG. 2 shows a schematic representation of a CSCT acquisition geometry along the rotational axis according to an exemplary embodiment of the present invention (after rebinning). The acquisition geometry depicted in FIG. 2 comprises a single-row energy-resolved detector system 37 comprising a first detecting element 42 and a second detecting element 43 which acquire data under the same scatter angle. The detector system 37 is, according to the exemplary embodiment depicted in FIG. 2, adapted in form of a focus-centred system in the xy-plane. However, it should be noted, that the detector system 37 may comprise many more single detecting elements, but for clarity reasons only detecting elements 42 and 43 are (schematically) depicted.

A polychromatic x-ray source 4 rotates around rotational axis 40 in a plane of rotation 41 and emits the x-ray beam to an object of interest, which is symbolized by circle 44. The object of interest 44 comprises a plurality of objects points 31, 32, 33, 34 and 35. During penetration of the object of interest 44, the electromagnetic radiation is scattered at the object points 31, 32, 33, 34 and 35. After rebinning, these object points are arranged along a line 36 which is perpendicular to a central ray 45 of the beam.

A first ray of radiation 46 corresponds to source position 412 and is scattered at the first point of interest 34 under a first scatter angle towards the first detecting element 42. Furthermore, a second ray of radiation 47 corresponds to source position 413 and is coherently scattered from the second object point 35 under a second scatter angle towards the second detecting element 43. Furthermore, a third ray of radiation 45 corresponding to source position 411 is scattered at the third point of interest 33 under a third scatter angle.

FIG. 3 depicts a schematic representation of the CSCT acquisition geometry of FIG. 2 in a fan-beam geometry for determining the detector geometry according to the present invention. Perpendicular line 36 (of FIG. 2) corresponds to circular arc 50 with its centre halfway between the source 411 and rotational axis 40 and with a diameter equal to the radius of the source path.

The first ray of radiation 46 is emitted under a first fan angle 391 and scattered at the first object point 341, which is, in fan-beam geometry, positioned on circuit arc 50. Accordingly, the second ray of radiation 47 is emitted under a second fan angle 392 and scattered at the second object point 351, which is, in fan-beam geometry, positioned on circuit arc 50.

FIG. 4 shows a schematic representation of the CSCT acquisition geometry of FIG. 2, but perpendicular to the rotational axis 40. As may be seen from FIG. 4, the detector array 37 is not only bend in the plane of rotation (see FIG. 2), but also in a plane perpendicular to the plane of rotation. Thus, the distance 48 between the first detecting element 42 and the plane of rotation 41 is different to the distance 49 of the second detecting element 43 and the plane of rotation 41. Advantageously, the distance depends on the position of the respective detecting element in the plane of rotation 41 and on the respective fan angle. In other words, the shape of the detector array 37 is bend in two directions dependent on whether the radiation detector 37 is a focus-centred single-row energy-resolved detector or a planar single-row energy-resolved detector. The double bending is such that all the scatter angles of the radiation scattered by object points 31, 32, 33, 34 and 35 are equal.

This automatically leads to a Cartesian q-sampling on a subsequently parallel-rebinned detector. Therefore, q-interpolation prior to the filtered back-projection reconstruction is avoided.

The basic method of filtered back-projection reconstruction for coherent scatter computed tomography has been described in U. van Stevendaal, J.-P. Schlomka, A. Harding, and M. Grass “A reconstruction algorithm for coherent scatter computed tomography based on filtered back-projection”, Med. Phys. 30 (9) (2003) pp. 2465-2474, which is hereby incorporated by reference.

FIG. 5 shows a flow-chart of an exemplary embodiment of a method according to the present invention. The method starts at step S1 with an acquisition of a projection data set. This may, for example, be performed by using a suitable CSCT scanner system or by reading the projection data from a storage. After that, in step S2, electromagnetic radiation is detected, which is coherently scattered from a first object point of the object of interest under a first scatter angle, by a first detecting element. At the same time, or before, or after that, electromagnetic radiation coherently scattered from a second object point of the object of interest under a second scatter angle is detected by a second detecting element. The first and the second detecting elements are arranged such that the first scatter angle equals the second scatter angle, wherein the first object point and the second object point are positioned on a line perpendicular to a central ray of the beam of electromagnetic radiation. First and second detecting elements may be part of a single-row radiation detector, for example a focus-centred single-row energy-resolved detector or a planar single-row energy-resolved detector. The distances between the plane of rotation and to the first detecting element or the second detecting element are a predetermined function of the first fan-angle between the central ray and a ray emitted from the source to the first object point and a function of a second fan-angle between the central ray and a ray emitted from the source to the second object point, respectively.

In a further step, a linear sampling in an energy for each detector element is performed and a parallel-beam rebinning of the beam into a parallel-beam geometry is applied, which may result in an equidistant sampling in an momentum transfer of the detected radiation for each detecting element without interpolation.

This may become clear by the following:

In case of a CSCT system with a single-row detector, which performs an energy-resolved measurement of the scattered photons, photons originating from the same line through the object of interest (perpendicular to the central ray of the fan) are measured under a different scatter angle, if each detector element in the row has the same distance to the plane of rotation. In order to compensate for this effect, a single-row detector with variable distance H (β) of the detector elements with respect to the plane of rotation may be used, according to an aspect of the present invention. This may automatically lead to a Cartesian q-sampling on a parallel rebinned detector and avoid the q-interpolation during the reconstruction. In case that an energy-resolved focus-centred single-row detection system is used (as depicted in FIGS. 2 and 3), the scatter angle Φ from object points on a line (perpendicular to the central beam of x-rays in the rotation plane) varies with the fan angle β (which lies inside the interval [−β₀;+β₀]) according to

$$Θ ( β ) = arctan ( \frac{H ( β )}{G - S cos ( β )} ) , \qquad ( 1 )$$

with G and S being the distance from the source to the detector and to the centre of rotation, respectively. The momentum transfer q is related to the scatter angle and the photon energy according to

$$q = \frac{E}{hc} sin ( \frac{Θ ( β )}{2} ) . \qquad ( 2 )$$

E is the energy of the photon while h and c mark Planck's constant and the velocity of the light.

Consequently, the bending of the single-row detector with respect to its distance from the plane of rotation H(β) must be
H(β)=tan Φ(G−S cos(β)), (3)

for a Φ value of interest and a focus-centred detector. Using this detector with bending in two directions, an equidistant sampling in q-direction automatically results, when a linear sampling in the energy E is used and a fan-beam to parallel-beam rebinning is applied. For a planar single-line energy-resolved detector, the relation between the scatter angle Φ and the fan-angle β is

$$Θ ( β ) = arctan ( \frac{H cos ( β )}{G - S {cos}^{2} ( β )} ) , \qquad ( 4 )$$

and the required bending in the distance to the plane of rotation results as
H(β)=tan θ (5)

to achieve the same effect.

For single-line detectors of different shape this technique may also be applied in order to reduce the loss in spatial resolution due to interpolation q-direction.

In other words, in energy-resolved coherent scatter CT the shape of the detector is optimized in order to derive an equidistant q-sampling. In general, the one-dimensional energy-resolved measurements are recalculated to a q-dimensional detector array of varying angle and q-value. Hence, the shape modification yields an optimal shape of the two-dimensional measurement space by deforming a one-dimensional array with equidistant energy sampled detector. Therefore, the shape of a one-dimensional detector is modified in order to achieve an optimal sample two-dimensional measurement space.

Advantageously, the detector is used in its full energy range. No upper and lower energy limits are used as a function of the fan-angle.

FIG. 6 depicts an exemplary embodiment of a data processing device according to the present invention for executing an exemplary embodiment of a method in accordance with the present invention. The data processing device depicted in FIG. 6 comprises a central processing unit or image processor 151 connected to a memory 152 for storing an image depicting an object of interest. The data processor 151 may be connected to a plurality of input/output network or diagnosis devices, such as a CSCT apparatus. The data processor may furthermore be connected to a display device 154, for example, a computer monitor, for displaying information or an image computed or adapted in the data processor 151. An operator or user may interact with the data processor 151 via a keyboard 155 and/or other output devices, which are not depicted in FIG. 6.

Furthermore, via the bus system 153, it may also be possible to connect the image processing and control processor 151 to, for example, a motion monitor, which monitors a motion of the object of interest. In case, for example, a lung of a patient is imaged, the motion sensor may be an exhalation sensor. In case, the heart is imaged, the motion sensor may be an electrocardiogram.

The acquisition geometry according to the present invention improves the spatial resolution and the computational efficiency in CSCT reconstruction, since one interpolation must not be carried out during the pre-processing of the reconstruction. This invention disclosure is important for coherent scatter computer tomography for medical applications and baggage inspection (new business).

It should be noted, that the term “comprising” does not exclude other elements or steps and the “a” or “an” does not exclude a plurality and that a single processor or system may fulfil the functions of several means recited in the claims. Also elements described in association with different embodiments may be combined.

It should also be noted, that any reference signs in the claims shall not be construed as limiting the scope of the claims.

1. A computer tomography apparatus for examination of an object of interest, the computer tomography apparatus comprising:
a rotating source (4) of electromagnetic radiation emitting a beam of electromagnetic radiation to an object of interest (7);
a first detecting element adapted for detecting electromagnetic radiation coherently scattered from a first object point (341) of the object of interest (7) under a first scatter angle;
a second detecting element adapted for detecting electromagnetic radiation coherently scattered from a second object point (351) of the object of interest (7) under a second scatter angle;
wherein the first object point (341) and the second object point (351) are positioned on a circular arc (50);
wherein the first scatter angle equals the second scatter angle; and
wherein the first detecting element and the second detecting element are part of a single-row energy-resolved detector.

2. The computer tomography apparatus of claim 1,
wherein a first distance between the first detecting element and a plane of rotation of the rotating source (4) of electromagnetic radiation is a predetermined function of a first fan angle between the central ray and a ray emitted from the source (4) to the first object point (341); and
wherein a second distance between the second detecting element and the plane of rotation is a predetermined function of a second fan angle between the central ray and a ray emitted from the source (4) to the second object point (351).

3. The computer tomography apparatus of claim 1, further comprising a data processor, wherein the data processor (151) is adapted for performing the following operation:
linear sampling in an energy for each detecting element; and
applying a parallel-beam rebinning of the beam into a parallel beam geometry, resulting in an equidistant sampling in an momentum transfer of the detected radiation for each detecting element without interpolation.

4. The computer tomography apparatus of claim 1,
wherein the first detecting element and the second detecting element are part of a radiation detector; and
wherein the radiation detector is one of a focus-centred single-row energy-resolved detector and a planar single-row energy-resolved detector.

5. The computer tomography apparatus of claim 1,
wherein the source (4) of electromagnetic radiation is a polychromatic x-ray source;
wherein the source (4) moves along a helical path around the object of interest (7); and
wherein the beam has a fan-beam geometry.

6. The computer tomography apparatus of claim 1, being adapted as a coherent scatter computer tomography apparatus.

7. The computer tomography apparatus of claim 1, configured as one of the group consisting of a baggage inspection apparatus, a medical application apparatus, a material testing apparatus and a material science analysis apparatus.

8. A radiation detector comprising:
a first detecting element adapted for detecting electromagnetic radiation emitted from a rotating source (4) of electromagnetic radiation and coherently scattered from a first object point (341) of an object of interest (7) under a first scatter angle;
a second detecting element adapted for detecting electromagnetic radiation emitted from the source (4) and coherently scattered from a second object point (351) of the object of interest (7) under a second scatter angle;
wherein the first object point (341) and the second object point (351) are positioned on a circular arc (50);
wherein the first scatter angle equals the second scatter angle; and
wherein the first detecting element and the second detecting element are part of a single-row energy-resolved detector.

9. The radiation detector of claim 8,
wherein a first distance between the first detecting element and a plane of rotation of the rotating source (4) is a predetermined function of a first fan angle between the central ray and a ray emitted from the source (4) to the first object point (341); and
wherein a second distance between the second detecting element and the plane of rotation is a predetermined function of a second fan angle between the central ray and a ray emitted from the source (4) to the second object point (351).

10. The radiation detector of claim 8,
wherein a linear sampling in an energy for each detector element and an application of a parallel-beam rebinning of the beam into a parallel beam geometry results in an equidistant sampling of an momentum transfer of the detected radiation for each detecting element without interpolation.

11. The radiation detector of claim 8,
wherein the radiation detector is one of a focus-centred single-row energy-resolved detector and a planar single-row energy-resolved detector.

12. A method of examination of an object of interest (7) in a computer tomography apparatus, the method comprising the steps of:
rotating a source (4) of electromagnetic radiation;
emitting a beam of electromagnetic radiation from the source (4) to an object of interest (7);
detecting electromagnetic radiation coherently scattered from a first object point (341) of the object of interest (7) under a first scatter angle by a first detecting element;
detecting electromagnetic radiation coherently scattered from a second object point (351) of the object of interest (7) under a second scatter angle by a second detecting element;
wherein the first object point (341) and the second object point (351) are positioned on a circular arc (50);
wherein the first scatter angle equals the second scatter angle; and
wherein the first detecting element and the second detecting element are part of a single-row energy-resolved detector.

13. The method of claim 12,
wherein a first distance between the first detecting element and a plane of rotation of the source (4) is a predetermined function of a first fan angle between the central ray and a ray emitted from the source (4) to the first object point (341);
wherein a second distance between the second detecting element and the plane of rotation is a predetermined function of a second fan angle between the central ray and a ray emitted from the source (4) to the second object point (351);
wherein the first detecting element and the second detecting element are part of a radiation detector; and
wherein the radiation detector is one of a focus-centred single-row energy-resolved detector and a planar single-row energy-resolved detector.

14. The method of claim 12, further comprising the steps of:
linear sampling in an energy for each detector element; and
applying a parallel-beam rebinning of the beam into a parallel beam geometry, resulting in an equidistant sampling in an momentum transfer of the detected radiation for each detecting element without interpolation.

15. A computer program for performing an examination of an object of interest (7) in a computer tomography apparatus, wherein the computer program causes a processor to perform the following operation when the computer program is executed on the processor:
loading a data set acquired by means of a rotating source (4) of electromagnetic radiation emitting a beam of electromagnetic radiation to an object of interest (7), the data set comprising:
first data detected by a first detecting element and corresponding to electromagnetic radiation coherently scattered from a first object point (341) of the object of interest (7) under a first scatter angle; and
second data detected by a second detecting element and corresponding to electromagnetic radiation coherently scattered from a second object point (351) of the object of interest (7) under a second scatter angle;
wherein the first object point (341) and the second object point (351) are positioned on a circular arc (50);
wherein the first scatter angle equals the second scatter angle; and
wherein the first detecting element and the second detecting element are part of a single-row energy-resolved detector.
